(19) United States
(12) Patent Application Publication
(10) Pub. No.: US 2008/0228068 A1
(43) Pub. Date: Sep. 18, 2008

(54) Automated Surgical Navigation with Electro-Anatomical and Pre-Operative Image Data

(76) Inventors: Raju R. Viswanathan, St. Louis, MO (US); Walter M. Blume, St. Louis, MO (US); Oren Liav, Nesher (IL); Zafrir Patt, Ramat-Hasharon (IL); Assaf Rubissa, Misgav (IL)

Correspondence Address: HARNESS, DICKEY, & PIERCE, P.L.C, 7700 Bonhomme, Suite 400, ST. LOUIS, MO, 63105, US

(21) Appl. No.: 11/685,664

(22) Filed: Mar. 13, 2007

(51) Int. Cl. A61B 5/00; A61B 18/12

(52) U.S. Cl. 600/426; 606/41

(57) ABSTRACT

A method for navigating a medical device to an anatomical surface within a subject to perform electro-anatomical mapping using a magnetic navigation system is provided that includes importing a pre-operative image data set of an anatomical surface in the subject's body into a localization system. One or more control parameters are applied to the magnetic navigation system to drive the medical device to one or more points of tissue contact, from the locations of which a geometric anatomical map can be created and registered with the pre-operative anatomical image. The pre-operative anatomical surface image and a representation of the geometric anatomical map are displayed relative to one another, such that a user may select a location on the displayed pre-operative anatomical image to navigate the medical device towards. The localization system provides the location data to the magnetic navigation system to drive the medical device to the desired location, for enabling further electrophysiology mapping or ablation treatment.

### FIELD

The present disclosure relates to magnetic navigation systems that remotely actuate medical devices, and in particular to methods for navigating medical devices to map and/or treat anatomical surfaces with a subject's body.

### BACKGROUND

The statements in this section merely provide background information related to the present disclosure and may not constitute prior art.

Medical procedures such as minimally interventional diagnosis and treatment of cardiac arrhythmias in electrophysiology often involve steering a localized medical device such as a catheter within anatomical regions in order to create a geometrical representation or map of the anatomical chamber of interest. In such a procedure, a localized catheter is steered to various sites within the anatomical chamber, and the three dimensional coordinates at each such location are recorded by a localization system after confirming that the device is indeed in contact with an internal wall, thereby providing data for the creation of a geometric map of the internal surface of the chamber. The catheter is also equipped with ECG recording electrodes, which provide for confirming wall contact and also for sensing electrical signals to help create a map of electrical activity across the heart surface, where such a map can have in excess of 80 or 100 contact points. This type of procedure is commonly performed by hand with a manually steered catheter, and can be a laborious process.

### SUMMARY

The present disclosure relates to interventional electro-physiology (EP) procedures involving the navigation of a medical device to an anatomical surface within a subject's body, such as a heart wall for example, to perform electro-anatomical mapping and ablation on portions of the anatomical surface. In the various embodiments, a method for navigating a medical device within a subject's body is provided that includes importing a pre-operative three-dimensional data set of an anatomical surface in a subject's body within a localization system for monitoring spatial location of the medical device. By applying one or more navigational control parameters to the navigational system to drive the medical device to one or more points of contact with a heart tissue surface, and recording the three-dimensional location and sensed electrical activity associated with each point of contact, a geometric anatomical map can be created and registered with the pre-operative three-dimensional anatomical surface data set. A display device displays an image of the pre-operative three-dimensional anatomical surface and a representation of the geometric anatomical map, such that a user may select at least one other desired location on the displayed pre-operative anatomical surface to navigate the medical device towards. The navigation system then drives the medical device to the at least one other desired location.

In one embodiment, a method for navigating a medical device within a subject's body is provided that comprises the integration of both a navigation system and a localization system for respectively guiding and monitoring location of a medical device within a subject's body. The method includes importing a pre-operative three-dimensional data set of an anatomical surface within the subject's body into a localization system for monitoring spatial location of the medical device. The navigation system applies one or more navigational control parameters for driving the medical device relative to the pre-operative anatomical surface to one or more points of contact with the actual anatomical surface within the subject's body. The method then creates a geometric anatomical map from the three-dimensional location and sensed electrical activity associated with each of the one or more points of contact, and registers the geometric anatomical map with the pre-operative anatomical surface data. At least one other desired location is selected from the pre-operative anatomical surface, and localization system data is used to provide location data to the navigation system for driving the medical device to the at least one other desired location. The method updates the geometric anatomical map to include the additional location data and sensed electrical activity associated with the at least one other desired location.

In another aspect of the disclosure, a display device is preferably used to display a representation of the geometric anatomical map including the one or more points of contact, with the pre-operative anatomical surface data. The displayed representation of a geometric anatomical map is preferably an electro-anatomical map that displays the one or more points of contact, and the propagation of electrical activity along the electro-anatomical map. The user may select the at least one other desired location by moving a user input device to move a cursor being displayed on the image of the anatomical surface. The user may also identify a region on the displayed anatomical surface, to which the medical device may be driven to contact one or more desired locations within the region for mapping an outline of a defect within the identified region. A sequence of one or more contact points may be used to define design lines that encircle a target area on the anatomical surface, which may be used in ablating the tissue surface at or around the target area. The target area may be a scar region on a heart tissue surface, for example, and an outline of the scar region may be ablated by the medical device to provide treatment through electrical isolation of the scar tissue.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a flowchart illustrating a method for controlling the navigation of a medical device within a subject's body using electro-anatomical data and pre-operative anatomical surface data, according to the principles of the present disclosure.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The present disclosure relates to interventional electro-physiology (EP) procedures involving the navigation of a medical device to an anatomical surface within a subject's body, such as a heart wall for example, to perform electro-anatomical mapping and ablation on portions of the anatomical surface. In one embodiment, a method for navigating a medical device within a subject's body is provided that comprises the integration of both a navigation system and a localization system for respectively guiding and monitoring location of a medical device within a subject's body. The method includes importing a pre-operative three-dimensional data set of an anatomical surface within the subject's body into a localization system for monitoring spatial location of the medical device. The navigation system applies one or more navigational control parameters for driving the medical device relative to the pre-operative anatomical surface to one or more points of contact with the actual anatomical surface within the subject's body. The method then creates a geometric anatomical map from the three-dimensional location and sensed electrical activity associated with each of the one or more points of contact, and registers the geometric anatomical map with the pre-operative anatomical surface data. At least one other desired location is selected from the pre-operative anatomical surface, and localization system data is used to provide location data to the navigation system for driving the medical device to the at least one other desired location. The method updates the geometric anatomical map to include the additional location data and sensed electrical activity associated with the at least one other desired location.

A display device is preferably used to display a representation of the geometric anatomical map including the one or more points of contact, with the pre-operative anatomical surface data. The displayed representation of a geometric anatomical map is preferably an electro-anatomical map that displays the one or more points of contact, and the propagation of electrical activity along the electro-anatomical map. The user may select the at least one other desired location by moving a user input device to move a cursor being displayed on the image of the anatomical surface. The user may also identify a region on the displayed anatomical surface, to which the medical device may be driven to contact one or more desired locations within the region for mapping an outline of a defect or electrical activity abnormality within the identified region. A sequence of one or more contact points may be used to define design lines that encircle a target area on the anatomical surface, which may be used in ablating the tissue surface at or around the target area. The target area may be a scar region on a heart tissue surface, for example, and an outline of the scar region may be ablated by the medical device to electrically isolate the scar tissue.

In one embodiment of a method for navigating a medical device within a subject's body, the method generally includes importing a pre-operative three-dimensional data set of an anatomical surface in a subject's body within a localization system for monitoring spatial location of the medical device. By applying one or more navigational control parameters to the navigational system to drive the medical device to one or more points of contact with a heart tissue surface, and recording the three-dimensional location and sensed electrical activity associated with each point of contact, a geometric anatomical map can be created and registered with the pre-operative three-dimensional anatomical surface data set. A display device displays an image of the pre-operative three-dimensional anatomical surface and a representation of the geometric anatomical map, such that a user may select at least one other desired location on the displayed pre-operative anatomical surface to navigate the medical device towards. The navigation system then drives the medical device to the at least one other desired location.

In the various embodiments, methods for automatically navigating a medical device to specific desired locations within a patient's cardiac anatomy are provided which use the integration of a surgical navigation system with a localization system. The surgical navigation system automatically manipulates and guides the device within the patient, using feedback of the device position and orientation provided by the localization system. A preoperative three dimensional data set is available and registered to the localization system. This dataset provides further guidance for the surgical navigation system. The medical device is used both to acquire cardiac electrical signals for creating electro-physiology mapping information, as well as to deliver treatment in the form of ablations to cardiac tissue. An example of a system that helps create an electrophysiology map is the CARTO™ EP Mapping system manufactured by Biosense Webster Inc., wherein the system renders a continuous interpolated surface given a discrete set of “visited” interior or internal surface points as input.

Electro-anatomical mapping and ablation is an important part of interventional Electro-Physiology (EP) procedures, where the mapping serves a diagnostic purpose prior to application of Radio Frequency (RF) ablation therapy. The mapping process is based on visiting a large number of sites or locations in the interior of a heart chamber (endocardial surface) with a catheter having integral electrodes capable of recording intracardiac ECG signals. This is performed with an EP mapping and localization system such as Biosense's CARTO™, which records catheter spatial location to high accuracy together with recorded local ECG information in order to create an electro-anatomical map of the endocardial surface using geometric reconstruction and interpolation techniques.

Typically the catheter is moved manually in this mapping process. However, new approaches are possible with the integration of the Biosense CARTO™ system with a magnetic navigation system such as the Stereotaxis NIOBE® system. The present disclosure describes new techniques for performing electro-anatomical mapping and ablation with such an integrated system.

Initially, in the setup phase the localization system is spatially registered with the magnetic navigation system, so that the catheter location is always known in magnetic navigation system coordinates. In the first step of the mapping process with the integrated system, a preoperative three dimensional image data set of the specific patient anatomy is loaded onto the localization system. Without loss of generality, we consider mapping of one of the chambers of the cardiac anatomy of a patient as an example. The magnetic navigation system applies a set of pre-defined magnetic field directions or “presets” to drive the catheter in various directions to contact the anatomical surface at various points to create a set of data points for three-dimensionally mapping the anatomical surface.

In one embodiment, a pre-defined control variable of the remote navigation system serves to align the distal end of the medical device to a pre-determined orientation or configuration. In the case of a magnetic navigation system that steers the device with an externally applied magnetic field, the pre-defined control variable is a field direction and magnitude that will steer or align a magnetically responsive element on the distal end of the medical device to an approximately known pre-determined direction. By controllably advancing the medical device using a number of preset directions, the medical device can be articulated to perform a sequence of mapping steps along the anatomical surface, starting from an approximately known anatomical position.

The magnetic navigation system applies a set of pre-defined magnetic field directions or “presets” to drive the catheter and extend the tip approximately in predefined directions until the forward movement of the catheter stops upon contacting the heart wall. Such “stopping” points can be identified by constantly monitoring the orientation and location of the catheter tip. These points of contact are acquired or stored on the CARTO™ system together with the associated electrical activity information. In the second step, the points acquired are used to create a geometric surface representation on the CARTO™ system. The surface can be color coded to incorporate electrical activity information, as is done on the CARTO™ system, thereby creating an electro-anatomical map. Among others, the map can display the propagation of electrical activity on the endocardial surface. This electro-anatomical surface map is registered to approximately match the surface of the imported preoperative three dimensional image data by a suitable mathematical fitting method, thereby creating a registration of the freshly obtained mapped surface to the preoperative image.

In the third step, the preoperative image can now be used to select further locations to drive the catheter to in order to acquire more anatomical points that can be used to enhance the reconstruction of the electro-anatomical surface. An example of a set of locations is a “design line” defined on the CARTO™ system, which interpolates a curve on the endocardial surface as the user moves a cursor along a portion of the electro-anatomical map on the CARTO™ system. With the integration of the magnetic navigation system and the localization system, one or more such locations may be selected on the preoperative image by the user on the latter system and sent to the former system. The magnetic navigation system can then drive the catheter to the user-selected target point(s) by closed-loop control methods whereby the catheter tip location data from the localization system is monitored and used to control the motion of the catheter so as to reach the desired target location, or until contact with the endocardial wall is made. Because of shifts in overall cardiac position and conformation, a location selected on the preoperative image data may not necessarily correspond to an actual endocardial position in the current, intraoperative patient anatomy, so that endocardial contact could in some cases be made even before the target location derived from the preoperative image data is reached.

As such new locations are visited by the catheter, electrical mapping data is acquired, the electro-anatomical map is updated and the registration with the preoperative image data can be refined, either automatically or as desired by the user.

In one embodiment of a Navigation system and method for Electro-anatomical mapping, the electro-anatomical map obtained in the second step could indicate a region of scar tissue where electrical activity is abnormal. For diagnostic purposes, a finer mapping of this area may be desired. In this case the scar is color-coded and its outline is visible on the surface of the preoperative image data. The process described in step three is used to refine the map within the local region corresponding to the scar on the preoperative image data, so that its outline can be accurately identified. One or more ablation contours can be defined as a sequence of one or more design lines (as detailed in step three above) that encircle the scar region. In one embodiment the contour is exported from the CARTO™ user interface to the magnetic navigation system so that the three dimensional contour information is available to the latter, while in another embodiment a desired target location that is chosen on the preoperative image data automatically becomes a “Go to” target (selected for example by a double mouse click or other User Interface selection tool) that the magnetic navigation system immediately and automatically steers the device towards.

In an alternate embodiment, an entire contour or path becomes a sequenced path for successively visiting a set of locations on the path. The contour is sent to the magnetic navigation system from the localization system, and the magnetic navigation system automatically steers the device to visit a series of closely-spaced locations successively on the path. Such automatically navigated contours can be used in the RF ablation treatment of Ventricular Tachycardia (VT) or Atrial Fibrillation (AF).

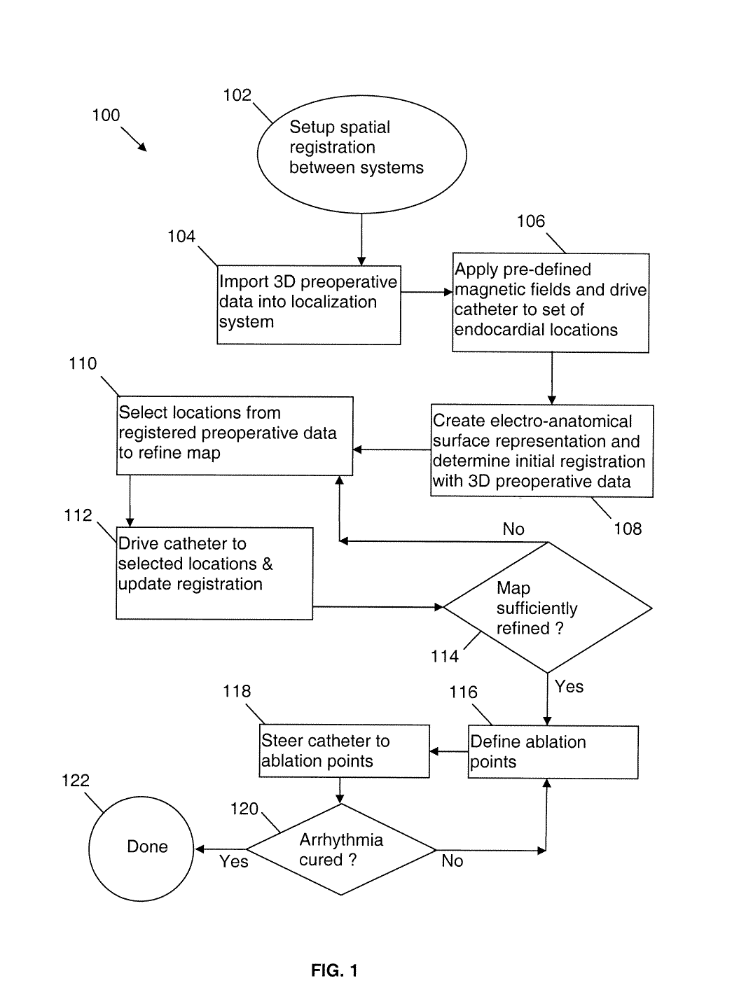

Referring to FIG. 1, a flowchart illustrating one embodiment of a method 100 for navigational control of a catheter device is shown. At step 102, the method initiates a spatial registration of a localization system that monitors the location of the catheter with the three-dimensional coordinates or frame of reference of a magnetic navigational system. A pre-operative three-dimensional image or data set of an anatomical surface in the subject's body is then imported into the localization system at step 104. The pre-operative anatomical surface may be displayed on a display device of the localization system. The magnetic navigation system then applies one or more pre-defined magnetic fields to drive the catheter to a set of locations or contact points on an endocardial surface at step 106. At step 108, the method then creates a geometric anatomical map using the three-dimensional location associated with each of the one or more points of contact, and registers the geometric anatomical map with the pre-operative anatomical surface data. The three-dimensional location and sensed electrical activity associated with each point of contact may also be recorded, and may displayed on the display device relative to the pre-operative image. At step 110, the user may select at least one other location on the displayed pre-operative anatomical surface to navigate the medical device towards, to further refine the geometric anatomical map. The localization system provides location data relating to the user-selected location to the navigation system, which drives the catheter to the selected locations at step 112. Step 114 repeats the user selection process in steps 110 and 112 until the map is sufficiently refined to allow for evaluation or diagnosis of the endocardial tissue. The user may then define ablation points at step 116, which the navigation system uses to steer the catheter to the ablation points at step 118. After ablation of the user selected points is complete, the catheter may be navigated to various points on the endocardial surface to verify whether an arrhythmia condition is still present at step 120. Steps 116 and 118 may accordingly be repeated until the desired outcome at step 120 is achieved.

The foregoing automated mapping methods and apparatus facilitate the quick creation of maps during medical procedures. Automated mapping is as fast as, or faster than, manual methods. Wasted movements are eliminated or minimized. The advantages of the above described embodiments and improvements should be readily apparent to one skilled in the art, as to enabling the navigation of interventional devices within a subject for mapping and ablation purposes. Additional design considerations may be incorporated without departing from the spirit and scope of the invention. Accordingly, it is not intended that the invention be limited by the particular embodiment or form described above, but by the appended claims.

What is claimed is:

1. A method for navigating a medical device within a subject's body with a magnetic navigation system and an electromagnetic localization system, the method comprising:
importing a pre-operative three-dimensional data set of an anatomical surface within the subject's body into a localization system for monitoring spatial location of the medical device;
applying one or more navigational control parameters to the magnetic navigation system for driving the medical device relative to the pre-operative anatomical surface to one or more points of contact with the actual anatomical surface within the subject's body;
creating a geometric anatomical map from the three-dimensional location and sensed electrical activity associated with each of the one or more points of contact;
registering the geometric anatomical map with the pre-operative anatomical surface data;
selecting at least one other desired location from the pre-operative anatomical surface and using localization system data to provide location data to the magnetic navigation system for driving the medical device to the at least one other desired location; and
updating the geometric anatomical map to include the location data and sensed electrical activity associated with the at least one other desired location.

2. The method of claim 1 further comprising the step of displaying a representation of the geometric anatomical map including the one or more points of contact, with the pre-operative anatomical surface data, on a display device.

3. The method of claim 2, wherein the displayed representation of a geometric anatomical map is an electro-anatomical map that displays the one or more points of contact and the propagation of electrical activity along the anatomical map.

4. The method of claim 3 wherein the at least one other selected point is selected by a user by moving a user input device to activate a cursor being displayed on the image of the anatomical surface.

5. The method of claim 3 further comprising the step of identifying a region on the displayed anatomical surface, such that the medical device may be driven to contact the one or more desired locations within the region, to map an outline of a defect within the identified region.

6. The method of claim 5 wherein the one or more points of contact and the at least one other desired location define a design line on the displayed image of the anatomical surface.

7. The method of claim 6 wherein a sequence of one or more design lines that encircle a target area on the anatomical surface may be defined for use in ablating the tissue surface.

8. The method of claim 1 wherein the device location data comprises device positional data and device orientational data.

9. The method of claim 1 wherein the position and orientation of the medical device are monitored by the localization system, and are used by the magnetic navigation system to control the movement of the medical device to guide the medical device to the desired location or until contact with the anatomical surface is made.

10. The method of claim 1, further comprising the step of recording the sensed electrical activity associated with the at least one other desired location, and updating the electro-anatomical map displayed relative to the pre-operative image.

11. A method for navigating a medical device within a subject's body with a magnetic navigation system and an electromagnetic localization system, the method comprising:
importing a pre-operative three-dimensional data set of an anatomical surface in a subject's body within a localization system for monitoring spatial location of the medical device;
applying one or more navigational control parameters to the navigational system for driving the medical device relative to the pre-operative anatomical surface to one or more points of contact with the actual anatomical surface within the subject's body;
recording the three-dimensional location and sensed electrical activity associated with each of the one or more points of contact;
displaying an image of the pre-operative three-dimensional anatomical surface on a display device;
registering a geometric anatomical map, created from the one or more points of contact, with the pre-operative anatomical surface;
displaying an image of the pre-operative three-dimensional anatomical surface on a display device, and a representation of the geometric anatomical map relative to the pre-operative anatomical surface, on a display device; and
selecting at least one other desired location on the displayed pre-operative anatomical surface to navigate the medical device towards.

12. The method of claim 11 further comprising the step of providing localization system data to the magnetic navigation system for driving the medical device to the at least one other desired location.

13. The method of claim 12 wherein the location of the medical device is monitored by the magnetic navigation system using location data from the localization system, and is used to control the movement of the medical device to guide the medical device to the desired location or until contact with the anatomical surface is made.

14. The method of claim 12, wherein the representation of a geometric anatomical map including the one or more points of contact is an electro-anatomical map that displays information relating to the propagation of electrical activity along the anatomical surface.

15. The method of claim 14, further comprising recording the sensed electrical activity associated with the at least one other desired location, and updating the electro-anatomical map displayed relative to the pre-operative image.

16. The method of claim 12 wherein the at least one other desired location is selected by a user moving a user input device to move a cursor being displayed on the image of the pre-operative anatomical surface.

17. The method of claim 12 further comprising the step of identifying a region on the displayed anatomical surface, such that the medical device may be driven to contact one or more desired locations within the region, to map an outline of a defect within the identified region.

18. The method of claim 12 wherein the at least one other desired location is defined from at least one design-line or curve graphically drawn by the user on the displayed image of the anatomical surface.

19. The method of claim 18 wherein a sequence of one or more design-lines that encircle a target area on the anatomical surface may be defined for use in ablating the tissue surface.

20. The method of claim 19 wherein the target area is a scar region on a heart tissue surface, and a contour corresponding to an outline of the scar region is ablated by the medical device.
